(19) United States
(12) Patent Application Publication
(10) Pub. No.: US 2021/0007627 A1
(43) Pub. Date: Jan. 14, 2021

(54) Mobile Device

(71) Applicants: Owlstone Medical Limited, Cambridge (GB); Owlstone Inc., Westport, CT (US)

(72) Inventors: Andrew H. Koehl, Westport, CT (US); Max Allsworth, Essex (GB)

(73) Assignees: Owlstone Medical Limited, Cambridge (GB); Owlstone Inc., Westport, CT (US)

(21) Appl. No.: 16/980,041

(22) PCT Filed: Mar. 12, 2019
(86) PCT No.: PCT/GB2019/050685

§ 371 Date: Sep. 11, 2020

(30) Foreign Application Priority Data
Mar. 12, 2018 (GB) 1803913.1

(51) Int. Cl. A61B 5/08; A61B 5/097; A61B 5/00

(52) U.S. Cl. CPC A61B 5/082; A61B 5/7475; A61B 5/6898; A61B 5/097

(57) ABSTRACT

Mobile device (110) is disclosed. The present techniques relate to a mobile device or portable electronic device, particularly a device including a breath sampling device. The mobile electronic device (110) comprises a collection device (112) for collecting and storing a gas sample. The collection device (112) is accessible to enable analysis of the gas stored in the collection device (112). The collection device (112) comprises a mouthpiece (118) into which a user exhales. The collection device (112) further comprises a plurality of sorbent tubes (114) to collect and store the breath sample(s). Each tube (114) has a one-way valve (116) to prevent captured breath escaping.

### TECHNICAL FIELD

The invention relates to a mobile device or portable electronic device, particularly a device including a breath sampling device.

### BACKGROUND

It is known to incorporate a chemical sensor in a mobile device such as a mobile phone or a tablet. One such example is shown in EP2816352 which describes a portable electronic device incorporating a chemical sensor which can be used for breath analysis, e.g. for determining the breath alcohol content or bad breath.

The applicant has recognised the need for an alternative solution for using mobile devices incorporating chemical sensors.

### SUMMARY

According to the present invention there is provided an apparatus as set forth in the appended claims. Other features of the invention will be apparent from the dependent claims, and the description which follows.

We describe a mobile electronic device comprising a collection device for collecting and storing a gas sample, wherein the collection device is accessible to enable analysis of the gas stored in the collection device.

A mobile electronic device is any portable device such as a mobile phone, a smart phone or a tablet. This is typically a device which a user carries with them most of the time. Accordingly, it is convenient for a user to be able to collect a gas sample using such a mobile device. The gas sample is typically not analysed immediately but is collected and stored for later analysis, for example by a third party service provider in a laboratory.

The collection device may be fixed to the mobile device. Alternatively, the collection device may be at least partially or wholly detachable or removable from the mobile device. In this way, it is easier to access the collection device to analyse the collected gas sample which is stored in the device.

The collection device may be housed within the mobile device. Where the collection device is housed within the mobile device, the analysis may take place with the collection device within the mobile device. The collection device may be removable from the mobile device and in this arrangement, the analysis may take place after the collection device has been removed from the mobile device.

The collection device may be external to the mobile device. Where the collection device is external to the mobile device, the analysis may take place with the collection device attached to the mobile device. The collection device may be detachable from the mobile device and in this arrangement, the analysis may take place after the collection device has been detached from the mobile device.

The collection device may be configured to store multiple gas samples. For example, the collection device may comprise a plurality of sorbent tubes, one for each gas sample. Alternatively, each sorbent tube may be configured to store a plurality of samples.

There may be at least one additional sensor for collecting information simultaneously with the collection device. For example, there may be a carbon dioxide sensor, a humidity sensor, a microphone, a camera, one or more movement sensors for the mobile device, e.g. one or more gyroscopes to measure tilt, acceleration etc.

The mobile device may further comprise a processor configured to initiate collection of the gas sample in response to an input. The input may be a user input on a user interface. The user input may be prompted by detection of an external event, e.g. using one or more of the additional sensors.

The processor may be further configured to: determine whether the collected gas sample satisfies collection criteria and if so, stop the collection and store the collected gas sample. The processor may be further configured to determine whether a plurality of gas samples are to be collected. If multiple gas samples are to be collected, the collection device may continue to collect samples until the collection criteria for the plurality of samples are met. The collection criteria may include a minimum or maximum threshold volume for each sample or the plurality of samples, a minimum time threshold and/or a maximum time threshold for collection of the sample(s).

The mobile device may further comprise a user interface. The processor may be configured to prompt a user to send the collection device for analysis. For example, the user may be prompted to send the collection device when the collection criteria are met. Alternatively, the user may be prompted to return the mobile device as part of an upgrade or periodic renewal program. In such an example, the sample may be analysed when the mobile device is returned to the third party service provider.

The collection device may be configured to collect and store a breath sample or an air sample using any suitable known device. For example the collection device may be a sorbent tube or a Tedlar® bag. Tedlar® bags are described for example in “On the use of Tedlar® bags for breath-gas sampling and analysis” by Beauchamp et al published in J Breath Res 2008 December. An example of a paper describing sorbent tubes is “Sorbent-based sampling methods for volatile and sem-volatile organic compounds in air Part 1: Sorbent based air monitoring options” by Woolfenden E published in J Chromatogr A 2010 April. Alternatively, or additionally, the collection device may be configured to collect airborne particulates, e.g. the collection device may be filter or may comprise a filter for airborne particulates. The collection device may thus be configured to collect adsorbed and/or absorbed gas samples.

We also describe a method of collecting and storing a gas sample comprising: initiating collection of the gas sample in a collection device on a mobile device; determining whether the collected gas sample satisfies collection criteria and when it is determined that the collection criteria are met, storing the collected gas sample. The features of the mobile device as described above also apply to the method.

We also describe storing data from a plurality of gas sample, the method comprising: collecting and storing a plurality of gas samples as described above, analysing each of the stored gas samples; and storing the results of the analysing The method may further comprise storing the analysis of each of the plurality of gas samples in a library or similar database, e.g. for later or further analysis. Each of the stored gas samples may be collected with a different mobile device. The analysis may be stored together with a record of the mobile device which collected the sample.

### BRIEF DESCRIPTION OF THE DRAWINGS

For a better understanding of the invention, and to show how embodiments of the same may be carried into effect, reference will now be made, by way of example only, to the accompanying diagrammatic drawings in which:

FIG. 1a is a schematic illustration of a mobile device with a collection device;

FIG. 1b is a block diagram of the components of the mobile device of FIG. 1a;

FIG. 2a is a flowchart of a first method of using a mobile device such as that shown in FIG. 1a;

FIG. 2b is a flowchart of a second method of using a mobile device such as that shown in FIG. 1a.

### DETAILED DESCRIPTION OF THE DRAWINGS

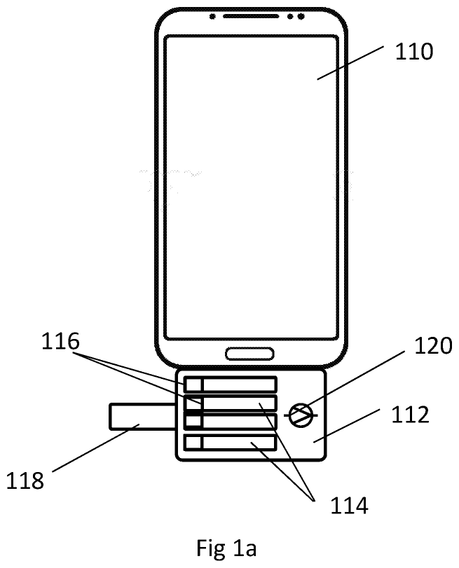

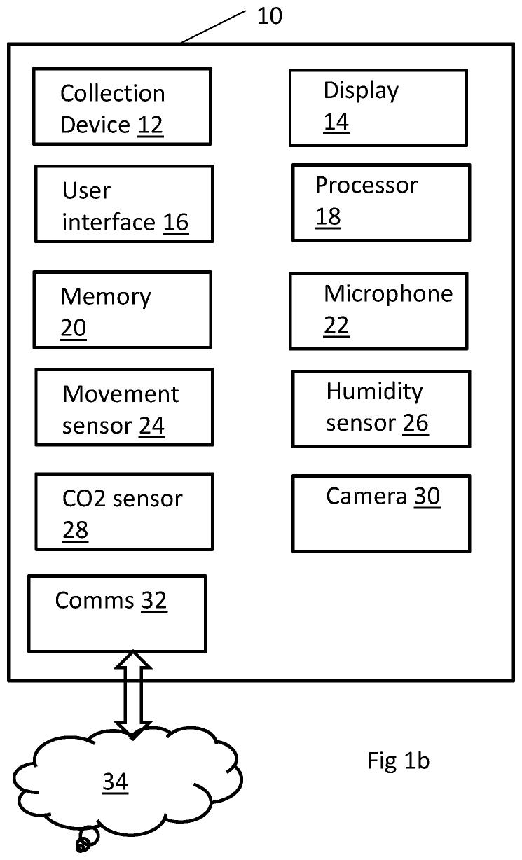

FIG. 1a shows a mobile electronic device 110 in the form of a mobile phone. It will be appreciated that a mobile phone is just one example of a mobile electronic device and other mobile electronic devices such as tablets may also incorporate a collection device. The mobile device 110 is connected to a collection device 112. In this example, the collection device is particularly adapted for breath analyser but it will be appreciated that the mobile device could receive other forms of collection device. Moreover, in this example, the collection device 112 is external to the mobile device 110 but it will be appreciated that the collection device 112 may be embedded within the housing of the mobile device 110.

The collection device 112 comprises a mouthpiece 118 into which a user exhales and a plurality of sorbent tubes 114 to collect and store the breath sample(s). Each tube 114 has a one-way valve 116 to prevent captured breath escaping. In this example, the collection device also comprises a pump 120. This draws ambient air into each sorbent tube 114 after each breath capture, e.g. for 10 seconds, so that multiple breath collections may be captured on a single tube 114 and spatially separated along the tube by use of an air gap between successive collections. Alternatively, each tube 114 may be used to capture a single breath. If each tube can only capture a single breath, the collection device 112 may only be used to capture as many breaths as there are tubes.

FIG. 1b shows a block diagram of the components of the mobile device 10 including a collection device 12. The mobile device 10 comprises the standard components of a mobile device, for example a display 14 (e.g. a screen) which displays information to a user and a user interface 16 through which a user can enter input and. The user interface may be any known user interface including a touch-sensitive screen, a keyboard or a voice activated interface. The mobile device 10 comprises a processor 18 which processes instructions from apps running on the device and memory 20 for storing information, including apps which have been downloaded. The mobile device 10 comprises a communication module 32 which allows the mobile device to connect to a third party storage, e.g. to the cloud 34, to access analysed data as described below.

The mobile device 10 may also optionally comprise additional sensors such as at least one microphone 22 to collect audio data including the sound of breathing or other sounds; at least one movement sensor 24, for example to capture any of the tilt, acceleration and/or other movement of the collection device; a humidity sensor 26, a carbon dioxide sensor 28 and/or a camera 30 to capture image data. These sensors may collect information simultaneously with the sample collected by the collection device. Alternatively, or additionally, these sensors may collect data which triggers the sample collection.

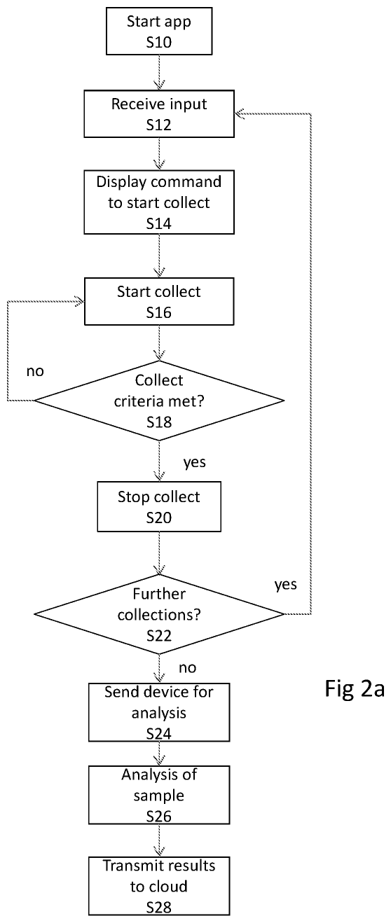

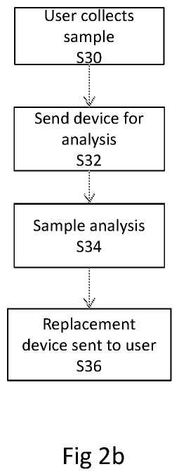

FIG. 2a shows a method of using a mobile device as described above. In a first step S10, the user launches an app which may have been pre-loaded on the mobile device. The user may launch the app, for example, because they wish to collect a sample. Once the app has been launched, the mobile device may detect an input (step S12). For example, the launch of the app causes a user interface to be displayed on the mobile device through which the user is able to make inputs. The input may not be generated by the user, for example an input may be a message from a third party, e.g. a healthcare professional. Alternatively, the input may be a detected event. For example, the microphone may detect a user coughing and the mobile device may be configured to determine whether or not the detected coughing is abnormal, e.g. more frequent than usual, and if so determine that an event has occurred. Similarly, the camera may capture an image and the mobile device may be configured to analyse the image to identify poor ambient air quality and if so determine that an event has occurred. As an another example, the carbon dioxide sensor may detect a level of carbon dioxide above a threshold. Another detected event may be a proximity signal when a user travels to certain locations (e.g. locations known to have poor air quality or known elevated levels of respiratory disease).

In response to the input, the mobile device displays a command to prompt a user to start capturing a sample (step S14). For example, the user interface can display a button labelled as “start collection” which can be selected or clicked by a user to initiate collection of samples by the collection device. It will be appreciated that a “start collection” button is just one way to present a user friendly interface.

The user input causes the collection device to automatically start collecting sample (step S16). For example, for a breath sample, the user exhales into the collection device for a predetermined amount of time, e.g. between 10 to 20 seconds, more particularly approximately 15 seconds. The predetermined amount of time may be determined based on the input and/or may be determined according to requirements prescribed by a healthcare professional. The collection device continues to collect the sample and periodically there is a determination as to whether or not the collected sample meets the required collection criteria (step S18). The collection criteria may comprise a minimum volume for the sample, a minimum time for the collection, e.g. the predetermined amount of time, and/or additional information from the mobile device, for example the position of the mobile device. If the sample does not meet the collection criteria, e.g. an insufficient quantity has been collected or the predetermined amount of time was not met, the collection device continues to collect the sample (S16) and then repeats the determining step. Once the collection criteria have been met, e.g. a large enough sample has been collected, the sample has been collected for a sufficient time duration and/or the position of the mobile device is no longer suitable for collecting a sample, the collection device stops collecting the sample (step S20).

If the collection device is suitable for the collection of multiple samples, a determination is made as to whether or not additional samples are to be collected. If further samples are to be collected, steps S12 to S20 are repeated. After either a prescribed number of sample collections (e.g. determined by the number of sorbent tubes as described above), a predetermined amount of time or as determined by the user's contract, the user is prompted to send the collection device for analysis. The collection device is then sent to a third party, e.g. someone who is not the user such as a specialist laboratory, the mobile device manufacturer or the mobile phone provider, for analysis (step S22). Where a separable collection device is used as described above, the user can remove the collection device and just send the collection device to the third party. Alternatively, if the collection device is integral with the mobile device so that the user is not able to detach the collection device from the mobile device, the mobile device with its integrated collection device is sent.

In either scenario, the collection device is then analysed (step S24). For an integrated device, this may require the third party to perform the analysis with the collection device in the mobile device or may require the third party to remove the collection device from the mobile device. The collection device may be completely removed and disconnected from the mobile device for analysis. Alternatively, the collection device may still be physically attached to the mobile device in such a way that it is accessible by the third party to perform the analysis.

The sample collected may be a user's breath and may be analysed for components which indicate disease. The analysis may be done in accordance with known techniques, e.g. those described in “Breath Analysis as a Potential and Non-Invasive Frontier in Disease Diagnosis: An Overview” by Periera et al published in Metabolites 2015, 5(1), 3-55. Alternatively, or additionally, the sample collected may be from the ambient environment and may be analysed for any suitable reason, e.g. to assess ambient quality or to determine contaminants to which a user has been exposed. The analysis can also involve analysing the collected sample alongside any other collected data, e.g. audio data such as the sound of breathing or other sounds, the tilt, acceleration and/or other movement of the collection device, humidity, levels of carbon dioxide and/or image data from a camera image. The results of the analysis are sent for storage, for example in the cloud (step S26). The results of either or both stages of analysis may also be sent to the user or may be accessed from the storage, e.g. via the app.

A removed collection device, whether removed by the user or the third party, may be reconditioned and returned to the mobile device or may be replaced by another collection device. A collection device which remains attached is preferably reconditioned before being reused. The replacement of the analysed collection device with a new or reconditioned collection device allows the user to continue to use the mobile device for collecting samples.

FIG. 2b is an alternative implementation for collecting samples using a collection device within a mobile device. Instead of the collection device being initiated and then controlled through the mobile device, the user controls the collection of samples in the collection device (step S30). The user also determines when the collected samples meet the collection criteria and then sends the collection device to the third party for analysis (step S32). The sample is then analysed (step S34) as described above. In this example, the collection device is a separate, detachable part which is connected to the mobile device and which is a single use device. Accordingly, the device cannot be used again and the user is sent a replacement device (step S36) to fit to the mobile device.

Although a few preferred embodiments of the present invention have been shown and described, it will be appreciated by those skilled in the art that various changes and modifications might be made without departing from the scope of the invention, as defined in the appended claims.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A mobile electronic device comprising a collection device for collecting and storing a gas sample, wherein the collection device is accessible to enable analysis of the gas stored in the collection device, wherein the mobile electronic device is one or more of a mobile phone, a smart phone and a tablet.

2. The mobile device of claim 1, wherein the collection device is detachable from the mobile device.

3. The mobile device of claim 1, wherein the collection device is housed within the mobile electronic device.

4. The mobile device of claim 1, wherein the collection device is configured to store multiple gas samples.

5. The mobile device of claim 1, further comprising at least one sensor for collecting information simultaneously with the collection device.

6. The mobile device of claim 1, further comprising a processor configured to initiate collection of the gas sample in response to an input.

7. The mobile device of claim 6, wherein the input is a user input.

8. The mobile device of claim 6, wherein the processor is further configured to:
determine whether the collected gas sample satisfies at least one collection criteria and
if so, stop the collection and
store the collected gas sample.

9. The mobile device of claim 6, wherein the processor is further configured to determine whether a plurality of gas samples are to be collected.

10. The mobile device of claim 6, further comprising a user interface and wherein the processor is configured to prompt a user to send the collection device for analysis.

11. (canceled)

12. The mobile device of claim 1, wherein the collection device is configured to collect and store a breath sample or an air sample.

13. The mobile device of claim 1, wherein the collection device is configured to collect a gas sample using one or both of adsorption or absorption.

14. A method of collecting and storing a gas sample comprising:
initiating collection of the gas sample in a collection device on a mobile device;
determining whether the collected gas sample satisfies collection criteria and
when it is determined that the collection criteria are met, storing the collected gas sample.

15. A method of storing data from a plurality of gas sample, the method comprising:
collecting and storing a plurality of gas samples as set out in claim 14;
analysing each of the stored gas samples; and
storing the results of the analysing.

16. The method of claim 15, wherein each of the stored gas samples is collected with a different mobile device.

17. The mobile device of claim 5, wherein the at least one sensor is used to trigger collection of the gas sample.

18. The mobile device of claim 5, wherein the at least one sensor is at least one of one or more microphones, one or more movement sensors, one or more humidity sensors, one or more carbon dioxide sensors, and one or more cameras.

19. The mobile device of claim 6, wherein the input is a detection of an external event using the at least one sensor.

20. The mobile device of claim 2, wherein the collection device is accessible by a third-party to enable analysis of the gas stored in the collection device when detached from the mobile electronic device.

21. The mobile device of claim 1, wherein the collection device is single use.
